(19) United States
(12) Patent Application Publication
(10) Pub. No.: US 2017/0224639 A1
(43) Pub. Date: Aug. 10, 2017

(54) 4-HYDROXY TAMOXIFEN GEL FORMULATIONS

(71) Applicant: BESINS HEALTHCARE LUXEMBOURG SARL, Luxembourg (LU)

(72) Inventor: Valerie Masini-Eteve, Bourg-La-Reine (FR)

(73) Assignee: BESINS HEALTH CARE LUXEMBOURG SARL, Luxembourg, (LU)

(21) Appl. No.: 15/293,845

(22) Filed: Oct. 14, 2016

Related U.S. Application Data
(63) Continuation of application No. 11/249,122, filed on Oct. 13, 2005, now abandoned.
(60) Provisional application No. 60/638,359, filed on Dec. 23, 2004.

(30) Foreign Application Priority Data
Oct. 14, 2004 (EP) 04 292 447.2

(51) Int. Cl. A61K 31/138; A61K 47/14; A61K 9/00; A61K 47/02; A61K 9/06; A61K 47/10; A61K 47/38

(52) U.S. Cl. CPC A61K 31/138; A61K 47/10; A61K 47/14; A61K 47/38; A61K 47/02; A61K 9/06; A61K 9/0014; A61K 9/0041

(57) ABSTRACT

Pharmaceutical compositions comprising 4-hydroxy tamoxifen in an amount of 0.105-0.950% by weight effectively deliver the 4-hydroxy tamoxifen. Such compositions, including gels, can treat or prevent benign breast disease, scarring or a keloid, gynecomastia, breast cancer, mastalgia, or a condition involving dense breast tissue.

### CROSS-REFERENCE TO RELATED PATENT APPLICATIONS

This application claims benefit of European patent application No. 04 292 447.2, filed Oct. 14, 2005, and claims benefit of U.S. patent application No. 60/638,359, filed Dec. 23, 2004. Both benefit applications, in their entirety, are incorporated herein by reference.

### FIELD OF THE INVENTION

The present invention relates to (4-hydroxy tamoxifen)-containing pharmaceutical compositions and gels, and to methods using the same.

### BACKGROUND OF THE INVENTION

The compound 4-hydroxy tamoxifen (hereinafter referred to as 4-OHT), or 1-[4-(2-N-dimethylaminoethoxy)phenyl]-1-(4-hydroxyphenyl)-2-phenylbut-1-ene, constitutes an active metabolite of the well characterized anti-estrogen compound, tamoxifen. Due to the presence of a double bond between two carbon atoms, 4-hydroxy tamoxifen exists in two stereoisomeric forms. According to the medical and biochemical literature, isomeric forms of 4-hydroxy tamoxifen are commonly designated as cis and trans isomers. From a purely chemical perspective, however, this designation is not strictly accurate because each double bonded carbon atom does not contain an identical chemical group. Therefore, it is more appropriate to refer to the isomers as (E) (the so-called cis form) and (Z) (the so-called trans form) configurations.

U.S. Pat. No. 4,919,937 to Mauvais-Jarvis et al. discloses 4-OHT formulations. However, these formulations do not contain any penetration enhancer, and are specifically designed for the co-administration of 4-OHT together with progesterone.

There is a need for improved 4-OHT containing formulations, which are easy to use for the patient, with easy compliance, especially for the delivery of higher doses, allow reproducible delivery of 4-OHT, with improved safety profiles and appropriate systemic effects. There is also a need for 4-OHT topical formulations, which allow efficient penetration of the active ingredient across the skin.

### SUMMARY OF THE INVENTION

The present invention provides 4-OHT-containing pharmaceutical compositions and gels, and methods for using the same. More particularly, the present invention provides hydroalcoholic pharmaceutical compositions suitable for topical and transdermal application, wherein said compositions contain 0.105-0.950% of 4-hydroxy tamoxifen by weight based on the total weight of the composition.

Compositions of the invention achieve a very effective 4-OHT delivery, alone or in combination with another medicament. Furthermore, compositions of the invention allow efficient patient compliance, in particular where higher doses are concerned (e.g., 1 mg 4-OHT or more, preferably 2 mg 4-OHT or more. Compositions of the invention also show appropriate systemic exposure.

### DETAILED DESCRIPTION OF THE PREFERRED EMBODIMENTS

The present invention thus relates to a pharmaceutical composition comprising 4-OHT, at least one C2-C6 alcohol, at least one gelling agent, at least one penetration enhancer, and an aqueous vehicle.

Said pharmaceutical composition can be made in various forms, e.g., a gel, a solution, a cream, a lotion, a spray, an ointment, an aerosol. Preferably, said pharmaceutical composition is a gel, and is suitable for topical administration.

The term “4-OHT” (4-hydroxy tamoxifen) as used therein refers to 1-[4-(2-N-dimethylaminoethoxy)phenyl]-1-(4-hydroxyphenyl)-2-phenylbut-1-ene. 4-OHT encompasses both the (Z) isomer and the (E) isomer, as well as mixtures thereof, including racemic and non racemic mixtures. The (Z) isomer is preferred because it is more active than the (E) isomer.

C2-C6 alcohols are known in the art. Such alcohols encompass ethanol, propanol, isopropanol (propan-2-ol), n-propanol (propan-1-ol), butanol, butan-1-ol, butan-2-ol, ter-butanol, pentanols, and hexanols. Ethanol is preferred, since it contributes efficiently towards the transdermal passage of 4-OHT by evaporating quickly on contact with the skin.

Gelling agents are known in the art. The term ‘gelling agent’ generally refers to a compound, possibly of polymeric nature, having the capacity to gel when contacted with a specific solvent, e.g., water. Gelling agents make it possible to increase the viscosity of the pharmaceutical compositions according to the invention, but may also act as solubilizing agents. Examples of gelling agents include anionic polymers such as acrylic acid based polymers (including polyacrylic acid polymers, e.g. CARBOPOL® by B.F. Goodrich Specialty Polymers and Chemicals Division of Cleveland, Ohio)., cellulose derivatives, poloxamers and poloxamines, more precisely, Carbomers or acrylic acid-based polymers, e.g. Carbopol® 980 or 940, 981 or 941, 1382 or 1382, 5984, 2984, 934 or 934P (Carbopol® are usually polymers of acrylic acid crosslinked with allyl sucrose or allylpentaerythritol), Pemulen TR1 ® or TR2 ®, Ultrez, Synthalen CR, etc.); cellulose derivatives such as carboxymethylcelluloses, hydroxypropylcelluloses, hydroxyethylcelluloses, ethylcelluloses, hydroxymethylcelluloses, hydroxypropylmethylcelluloses, and the like, and mixtures thereof; poloxamers or polyethylene-polypropylene copolymers such as Lutrol® grade 68 or 127, poloxamines and other gelling agents such as chitosan, dextran, pectins, and natural gums. All of these gelling agents, alone or in combination, may be used in the pharmaceutical composition according to the invention. Cellulosics, including carboxymethylcelluloses, hydroxypropylcelluloses, hydroxyethylcelluloses, ethylcelluloses, hydroxymethylcelluloses, hydroxypropylmethylcelluloses, and mixtures thereof, are particularly preferred in the context of the present invention.

Penetration enhancers are also known in the art. A ‘penetration enhancer’ is generally an agent known to accelerate the delivery of the drug or active principle through the skin. These agents also have been referred to as penetration accelerants, adjuvants, and absorption promoters. This class of agents includes those with diverse mechanisms of action including those which have the function of improving the solubility and diffusibility of the drug, and those which improve transdermal absorption by changing the ability of the stratum corneum to retain moisture, softening the skin, improving the skin's permeability, acting as penetration assistants or hair-follicle openers or changing temporarily the state of the skin such as the boundary layer. The penetration enhancer within the context of the present invention can be a functional derivative of a fatty acid, which includes isosteric modifications of fatty acids or non-acidic derivatives of the carboxylic functional group of a fatty acid or isosteric modifications thereof. In one embodiment, the functional derivative of a fatty acid is an unsaturated alkanoic acid in which the —COOH group is substituted with a functional derivative thereof, such as alcohols, polyols, amides and substituted derivatives thereof. The term “fatty acid” means a fatty acid that has four (4) to twenty-four (24) carbon atoms. Non-limiting examples of penetration enhancers include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C8 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; glycol ethers including diethylene glycol monoethyl ether; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; pyrrolidones and N-alkylpyrrolidone; terpenes; hydroxyacids; urea; essential oils; and mixtures thereof. Preferred examples include myristate isopropyl. All of these penetration enhancers can be used either alone or in combination, e.g., combination of two or three different penetration enhancers.

Aqueous vehicles are known in the art. According to another aspect of the invention, said aqueous vehicle comprises, besides water, ingredients useful in adjusting pH, for instance at least one buffering agent. Buffering agents, especially pharmacologically acceptable buffering agents, are known in the art. In one aspect, said aqueous vehicle comprises at least one buffer, preferably selected from the group consisting of citrate buffers, e.g., sodium citrate and/or potassium citrate; tris buffers, e.g., tris maleate; phosphate buffers, including Sorensen-type buffers, dibasic or monobasic phosphate, e.g., sodium dibasic or monobasic phosphate.

In another aspect, the pharmaceutical composition of the invention further comprises a base. Advantageously, said base is preferably pharmaceutically acceptable, and is preferably selected from the group consisting of triethanolamine, sodium hydroxide, ammonium hydroxide, potassium hydroxide, arginine, aminomethylpropanol or tromethamine, and mixtures thereof. Where the pH of said pharmaceutical composition is not optimized for transdermal administration, e.g., where said gelling agent comprises at least one acrylic acid-based polymer, said base contributes to the neutralization of said pharmaceutical composition, for topical application onto human skin. Furthermore, said base (neutralizer) allows optimum swelling of the polymer chains during the neutralization of the charges and the formation of polymer salts. Especially when said gelling agent comprises an acrylic acid-based polymer, said base preferably comprises triethanolamine. It also allows an optimum viscosity to be achieved in the pharmaceutical composition according to the invention. The skilled person would know how to choose a suitable amount of said base in the composition, especially with respect to the nature of said gelling agent present therein, and the alcohol content of the composition, in order to reach the desired final pH in the composition. For example, with carbomers and/or if there is a high alcohol content, one can use tromethamine and/or NaOH as a base, in amounts chosen as to reach the desired final pH in the composition. The base/gelling agent ratio preferably is between 10:1 and 0.1:1, more preferably between 7:1 and 0.5:1, and still more preferably between 4:1 and 1:1.

Unless otherwise stated, percentages (%) refer to amounts by weight based upon total weight of the composition. Amounts of alcohol(s) herein refer to absolute alcohol content, e.g., absolute ethanol content.

According to one aspect, said pharmaceutical composition comprises 0.105-0.950%, preferably 0.105-0.195%, more preferably 0.105-0.185%, more preferably 0.105-0.175%, more preferably 0.105-0.165%, more preferably 0.105-0.155%, more preferably 0.105-0.145%, more preferably 0.105-0.135%, more preferably 0.105-0.125%, more preferably 0.107-0.119%, more preferably 0.111-0.118%, more preferably 0.112-0.117%, still more preferably 0.113-0.116%, most preferably 0.114-0.115% of 4-OHT.

According to another aspect, said pharmaceutical composition comprises 0.205-0.950%, preferably 0.210-0.900%, more preferably 0.215-0.800%, even more preferably 0.220-0.750%, even more preferably 0.220-0.700%, even more preferably 0.220-0.600%, even more preferably 0.220-0.500%, still more preferably 0.220-0.400%, most preferably 0.220-0.350% of 4-OHT.

According to another aspect of the invention, said pharmaceutical composition comprises 40-80%, preferably 45-75%, more preferably 50-75%, still more preferably 55-75%, most preferably 60-75% of at least one alcohol.

According to another aspect of the invention, said C2-C6 alcohol is selected from the group consisting of ethanol, propan-1-ol and propan-2-ol, and mixtures thereof. Preferably said C2-C6 alcohol comprises ethanol.

According to another aspect of the invention, said pharmaceutical composition comprises 0.1-5.0%, preferably 0.15-4.5%, more preferably 0.2-4.0%, even more preferably 0.25-3.5%, even more preferably 0.3-3.0%, even more preferably 0.4-2.5%, still more preferably 0.5-2.0%, most preferably about 0.5-1.5% of at least one gelling agent.

According to another aspect of the invention, said gelling agent comprises at least one selected from the group of cellulosics, including cellulose esters and derivatives (cellulose derivatives such as carboxymethylcelluloses, hydroxypropylcelluloses, hydroxyethylcelluloses, ethylcelluloses, hydroxymethylcelluloses, hydroxypropylmethylcelluloses, and the like); and mixtures thereof. Preferably, said gelling agent is selected from carboxymethylcelluloses, hydroxypropylcelluloses, hydroxyethylcelluloses, ethylcelluloses, hydroxymethylcelluloses, hydroxypropylmethylcelluloses, and mixtures thereof. Most preferably, said gelling agent comprises hydroxypropylcellulose, e.g., Klucel.

According to another aspect of the invention, said pharmaceutical composition comprises 0.1-5.0%, preferably 0.15-4.5%, more preferably 0.2-4.0%, even more preferably 0.25-3.5%, even more preferably 0.3-3.0%, even more preferably 0.4-2.5%, still more preferably 0.5-2.0%, most preferably about 0.5-1.5% of at least one penetration enhancer.

According to another aspect of the invention, said pharmaceutical composition comprises 0.4-2.0%, preferably 0.5-1.9%, more preferably 0.5-1.8%, even more preferably 0.5-1.7%, even more preferably 0.5-1.6%, even more preferably 0.5-1.5%, even more preferably 0.6-1.4%, even more preferably 0.7-1.3%, still more preferably 0.8-1.2%, most preferably about 0.9-1.1%, or 1.0% of myristate isopropyl.

The compositions according to the invention may include several penetration enhancers.

According to another aspect of the invention, said pharmaceutical composition comprises 20-50%, preferably 20-40% of an aqueous vehicle.

According to another aspect of the invention, said aqueous vehicle preferably comprises at least one buffer, especially a phosphate buffer.

In another aspect of the invention, the pH of the pharmaceutical composition is preferably 7-11, more preferably 7.5-10.5, even more preferably 8.0-10.0, still more preferably 8.5-10.0, most preferably about 8.5-9.5.

In one embodiment, the present invention provides a pharmaceutical composition comprising:
0.205-0.950% (respectively 0.105-0.950%) of 4-OHT,
40-80% of a least one C2-C6 alcohol, preferably ethanol,
0.1-5.0% of at least one gelling agent, preferably hydroxypropylcellulose,
0.1-5.0% of at least one penetration enhancer,
20-50% of an aqueous vehicle.
In another embodiment, the present invention provides a pharmaceutical composition comprising:
0.210-0.900% (respectively 0.105-0.185%) of 4-OHT,
40-75% of a least one C2-C6 alcohol, preferably ethanol,
0.15-4.5% of at least one gelling agent, preferably hydroxypropylcellulose,
0.15-4.5% of at least one penetration enhancer,
20-40% of an aqueous vehicle.

In another embodiment, the present invention provides a pharmaceutical composition comprising:
0.215-0.800% (respectively 0.105-0.165%) of 4-OHT,
45-75% of a least one C2-C6 alcohol, preferably ethanol,
0.2-4.0% of at least one gelling agent, preferably hydroxypropylcellulose,
0.2-4.0% of at least one penetration enhancer,
20-40% of an aqueous vehicle.

In another embodiment, the present invention provides a pharmaceutical composition comprising:
0.220-0.750% (respectively 0.105-0.145%) of 4-OHT,
45-75% of a least one C2-C6 alcohol, preferably ethanol,
0.25-3.5% of at least one gelling agent, preferably hydroxypropylcellulose,
0.25-3.5% of at least one penetration enhancer,
20-40% of an aqueous vehicle.

In another embodiment, the present invention provides a pharmaceutical composition comprising:
0.220-0.700% (respectively 0.105-0.125%) of 4-OHT,
50-75% of a least one C2-C6 alcohol, preferably ethanol,
0.3-3.0% of at least one gelling agent, preferably hydroxypropylcellulose,
0.3-3.0% of at least one penetration enhancer,
20-40% of an aqueous vehicle.

In another embodiment, the present invention provides a pharmaceutical composition comprising:
0.220-0.600% (respectively 0.111-0.118%) of 4-OHT,
60-75% of a least one C2-C6 alcohol, preferably ethanol,
0.4-2.5% of at least one gelling agent, preferably hydroxypropylcellulose,
0.4-2.5% of at least one penetration enhancer,
20-40% of an aqueous vehicle.

In another embodiment, the present invention provides a pharmaceutical composition comprising:
0.220-0.500% (respectively 0.112-0.117%) of 4-OHT,
60-75% of a least one C2-C6 alcohol, preferably ethanol,
0.5-2.0% of at least one gelling agent, preferably hydroxypropylcellulose,
0.5-2.0% of at least one penetration enhancer,
20-40% of an aqueous vehicle,

In another embodiment, the present invention provides a pharmaceutical composition comprising:
0.220-0.400% (respectively 0.113-0.116%) of 4-OHT,
60-75% of a least one C2-C6 alcohol, preferably ethanol,
0.5-1.5% of at least one gelling agent, preferably hydroxypropylcellulose,
0.5-1.5% of at least one penetration enhancer,
20-40% of an aqueous vehicle.

In another embodiment, the present invention provides a pharmaceutical composition comprising:
0.220-0.350% (respectively 0.114-0.115%) of 4-OHT,
60-75% of a least one C2-C6 alcohol, preferably ethanol,
0.5-1.5% of at least one gelling agent, preferably hydroxypropylcellulose,
0.5-1.5% of at least one penetration enhancer,
20-40% of an aqueous vehicle.

In another embodiment, the present invention provides a pharmaceutical composition comprising:
0.205-0.950% (respectively 0.105-0.950%) of 4-OHT,
40-80% of a least one C2-C6 alcohol, preferably ethanol,
0.1-5.0% of at least one gelling agent, preferably hydroxypropylcellulose,
0.4-2.0% of myristate isopropyl,
20-50% of an aqueous vehicle.

In another embodiment, the present invention provides a pharmaceutical composition comprising:
0.210-0.900% (respectively 0.105-0.185%) of 4-OHT,
40-75% of a least one C2-C6 alcohol, preferably ethanol,
0.15-4.5% of at least one gelling agent, preferably hydroxypropylcellulose,
0.5-1.8% of myristate isopropyl,
20-40% of an aqueous vehicle.

In another embodiment, the present invention provides a pharmaceutical composition comprising:
0.215-0.800% (respectively 0.105-0.165%) of 4-OHT,
45-75% of a least one C2-C6 alcohol, preferably ethanol,
0.2-4.0% of at least one gelling agent, preferably hydroxypropylcellulose,
0.6-1.4% of myristate isopropyl,
20-40% of an aqueous vehicle.

In another embodiment, the present invention provides a pharmaceutical composition comprising:
0.220-0.750% (respectively 0.105-0.145%) of 4-OHT,
45-75% of a least one C2-C6 alcohol, preferably ethanol,
0.25-3.5% of at least one gelling agent, preferably hydroxypropylcellulose,
0.5-1.5% of myristate isopropyl,
20-40% of an aqueous vehicle.

In another embodiment, the present invention provides a pharmaceutical composition comprising:
0.220-0.700% (respectively 0.105-0.125%) of 4-OHT,
50-75% of a least one C2-C6 alcohol, preferably ethanol,
0.3-3.0% of at least one gelling agent, preferably hydroxypropylcellulose,
0.6-1.4% of myristate isopropyl,
20-40% of an aqueous vehicle.

In another embodiment, the present invention provides a pharmaceutical composition comprising:
0.220-0.600% (respectively 0.111-0.118%) of 4-OHT,
60-75% of a least one C2-C6 alcohol, preferably ethanol,
0.4-2.5% of at least one gelling agent, preferably hydroxypropylcellulose,
0.7-1.3% of myristate isopropyl,
20-40% of an aqueous vehicle.

In another embodiment, the present invention provides a pharmaceutical composition comprising:
0.220-0.500% (respectively 0.112-0.117%) of 4-OHT,
60-75% of a least one C2-C6 alcohol, preferably ethanol,
0.5-2.0% of at least one gelling agent, preferably hydroxypropylcellulose,
0.8-1.2% of myristate isopropyl,
20-40% of an aqueous vehicle,

In another embodiment, the present invention provides a pharmaceutical composition comprising:
0.220-0.400% (respectively 0.113-0.116%) of 4-OHT,
60-75% of a least one C2-C6 alcohol, preferably ethanol,
0.5-1.5% of at least one gelling agent, preferably hydroxypropylcellulose,
0.9-1.1% of myristate isopropyl,
20-40% of an aqueous vehicle.

In another embodiment, the present invention provides a pharmaceutical composition comprising:
0.220-0.350% (respectively 0.114-0.115%) of 4-OHT,
60-75% of ethanol,
0.5-1.5% of hydroxypropylcellulose,
0.4-2.0% of myristate isopropyl,
20-40% of an aqueous vehicle.

Additionally, said pharmaceutical composition may comprise usual pharmaceutical additives, including salt(s), emollient(s), stabilizer(s), antimicrobial(s), fragrance(s), and/or propellant(s). Said pharmaceutical composition may also include at least one further active ingredient.

The invention also provides a gel useful for transdermal or transcutaneous delivery comprising said pharmaceutical composition according to the invention. Thus, the invention is also directed to a 4-OHT-containing hydro-alcoholic gel.

According to another embodiment, the present invention provides a dose packet, unit dose packet or multiple dose packet containing said pharmaceutical composition or said hydroalcoholic gel. Advantageously, such conditioning of said pharmaceutical composition renders application easier for a patient. Thus, these forms of packaging can reflect the schedule of application, e.g., daily or weekly administration.

According to another embodiment, the invention provides a dispenser, e.g., with a hand pump or valve, containing said pharmaceutical composition or said hydroalcoholic gel. Such dispensers allow for flexibility in the administered dosage, as a function of the amount of composition to be applied.

According to one embodiment, said packets or dispensers can be accompanied by a notice giving instructions for the use thereof.

The pharmaceutical compositions, gels, packets and containers of the invention are useful for treating various conditions and/or disorders.

The term ‘treat’ or ‘treatment’ as used herein refers to any treatment of a mammalian condition, disorder, or disease associated with a depressive disorder, and includes, but is not limited to, preventing the condition, disorder, or disease from occurring in a subject which may be predisposed to the condition, disorder, or disease, but has not yet been diagnosed as having the condition, disorder, or disease; inhibiting the condition, disorder, or disease, for example, arresting the development of the condition, disorder, or disease; relieving the condition, disorder, or disease, for example, causing regression of the condition, disorder, or disease; or relieving the condition caused by the disease or disorder, for example, stopping the symptoms of the disease or disorder.

The term ‘prevent’ or ‘prevention,’ in relation to a condition, disorder, or disease, means no condition, disorder, or disease development if none had occurred, or no further condition, disorder, or disease development if there had already been development of the condition, disorder, or disease.

In particular, the pharmaceutical compositions, gels, packets and containers of the invention are useful for:

treating and/or preventing conditions involving dense breast tissue. High density breast tissues are a predictor of breast cancer risk, and compromises mammographic sensitivity, which is a major issue for cancer detection and diagnostic. Said dense breast tissue can be diffuse or nodular;

treating and/or preventing benign breast diseases. Benign breast disease generally refers to a constellation of common non-malignant aberrations in breast tissue. These aberrations include numerous lesions that have well-defined histological characteristics, and can be classified as proliferative or nonproliferative. Exemplary benign breast diseases treatable by the present methods include adenosis, cysts, duct ectasia, fibroadenoma, fibrosis, hyperplasia, metaplasia and other fibrocystic changes. Each of these diseases, often referred to as “changes” or “conditions” due to their prevalence, have well-defined histological and clinical characteristics. “Adenosis” refers to generalized glandular disease of the breast. It typically involves an enlargement of breast lobules, which contain more glands than usual. In “sclerosing adenosis,” or “fibrosing adenosis,” the enlarged lobules are distorted by scar-like fibrous tissue. “Cysts” are abnormal sacs filled with fluid or semi-solid material, and lined by breast epithelial cells, developing from lobular structures. They begin as excess fluid inside breast glands, but may grow to proportions that stretch surrounding breast tissue, causing pain. “Fibrocysts” are cystic lesions circumscribed by, or situated within, a conspicuous amount of fibrous connective tissue. “Duct ectasia” refers to a dilation of mammary ducts by lipid and cellular debris. Rupture of the ducts induces infiltration by granulocytes and plasma cells. “Fibroadenoma” refers to benign tumors that are derived from glandular epithelium and contain a conspicuous stroma of proliferating fibroblasts and connective tissue. “Fibrosis” simply refers to a prominence of fibrous tissue in the breast. “Hyperplasia” refers to an overgrowth of cells, where several layers of cells line the basal membrane, without tumor formation. Hyperplasia increases the bulk of mammary tissue. In “epithelial hyperplasia,” the cells lining breast ducts and lobules are involved, giving rise to the terms “ductal hyperplasia” and “lobular hyperplasia.” Based on a histological determination, hyperplasia may be characterized as “usual” or “atypical.” “Metaplasia” refers to a phenomenon in which a differentiated tissue of one type transforms into a differentiated tissue of another type. Metaplasia often results from an environmental change, and enables cells better to withstand the change;

preventing and/or treating scars, including excessive scarring, keloid scars and hypertrophic scars. “Excessive scar” or “excessive scarring” generally refers to overgrowths of dense fibrous tissue that result from abnormal wound healing. Excessive scars have grown larger than necessary for normal wound healing, and are characterized by overproduction of cells, collagen and/or proteoglycan. “Keloid scars” are excessive scars in which the dense fibrous tissue extends beyond the borders of the original wound or incision, and does not usually regress spontaneously. Determining whether a scar is a keloid can be difficult, since keloids often superficially resemble other hypertrophic scars. However, keloids have distinguishing histological features. One such feature is the collagen nodule, which contains a high density of fibroblasts and unidirectional collagen fibrils in a highly organized and distinct orientation. Additionally, keloids have a rich vasculature, a high mesenchymal cell density, and a thickened epidermal cell layer. “Hypertrophic scars” are excessive scars in which the dense fibrous tissue does not extend beyond the borders of the original wound or incision. They tend to be wider than necessary for normal wound healing to occur. Histologically, hypertrophic scars have more organized collagen fibers than keloids, and scant mucoid matrix. Hypertrophic lesions are characterized by randomly distributed tissue bundles consisting of uniaxially oriented extracellular matrix and cells;

treating gynecomastia. Gynecomastia is a common clinical condition, often presenting secondarily to an underlying disorder, representing the benign and sometimes painful proliferation of breast tissue in young boys and adult males;

preventing and/or treating breast cancer, especially non-invasive breast cancer;

treating mastalgia. Mastalgia, also called “mastodynia” or breast pain constitutes the most common breast problem for which women consult general medical practitioners. Its severity varies, but mastalgia can be so prolonged and intense as to interfere with normal daily activities, and even to disable afflicted individuals. Mastalgia can be classified according to three general sources of pain: (1) cyclical mammary pain, (2) non-cyclical mammary pain, and (3) extramammary pain. Cyclical mastalgia results from physiological breast enlargement, caused by estrogen-dependent vascular changes, during the luteal phase of the menstrual cycle, and affects a majority of premenopausal women. Cyclical mastalgia also can recur in postmenopausal women on estrogen replacement therapy, with a dose-dependent effect. “Non-cyclical mastalgia,” as its name suggests, refers to pain in the breast that is not related to the menstrual cycle. A number of conditions give rise to non-cyclical mastalgia, including sclerosing adenosis, Tietz's syndrome and, rarely, breast cancer. Finally, extramammary mastalgia includes breast pain that is projected to the breast from other sources, as occurs, for example, when a patient feels pain from muscles or ribs that underlie the breasts.

The present pharmaceutical compositions and gels can also be used in “combination therapy” with a further active agent.

In another aspect, the invention also relates to the use of the gel or the solution according to the invention for the preparation of a medicinal product for transdermal application for the treatment of at least one of the above-mentioned indications.

In another aspect, the present invention relates to a method of treatment.

In one embodiment, said method comprises the step of administering 4-OHT topically, to a subject in need thereof.

According to one aspect, the pharmaceutical composition or the gel of the present invention is applied on one breast, or both, or on the scar, depending upon the indication.

The 4-OHT dosage can be adjusted by one skilled in the art. When applied onto breast skin, this can be 0.50-3.0 mg/day/breast, preferably 0.60-2.5 mg/day/breast, more preferably 0.75-2.3 mg/day/breast, even more preferably 1.0-2.0 mg/day/breast.

Ideally, the unit dose of the composition (gel) is from about 0.5 to about 3.5 grams, which represents the approximate range of pharmacokinetic linearity with the inventive gels. It was found that above 3.5 grams, patient compliance in addition to pharmacokinetic variability become limiting. Moreover, with less than about 0.5 grams, the pharmacokinetics become non-linear. More preferably, the unit dose of gel is more than about 0.75 grams or even more than about 1 gram.

In the case of a scar, the dosage can be 0.25-3.0 μg/cm², preferably 0.5-2.5 μg/cm², more preferably 1.0-2.0 μg/cm².

The pharmaceutical compositions and methods of the invention are particularly useful for patients, especially women, to the extent that they allow an easy and reproducible compliance, especially where higher doses are concerned. By higher doses, it is herewith understood doses of 1 mg 4-OHT or more, preferably doses of 2 mg 4-OHT or more. For such higher doses of 4-OHT, the actual amount of gel delivered to the patient is more appropriate, irrespective of the skin surface area used for application (e.g., breast size). In other terms, to a very large extent, patient compliance will not depend upon the area of skin used for application (e.g., size of breast or scar). By contrast, with other less concentrated formulations, the dose actually delivered to the patient would depend on the skin area used for application, and thus jeopardize proper compliance.

In another aspect, the invention provides a process for preparing said pharmaceutical composition or gel according to the invention.

In one aspect, said process comprises the step of preparing a mixture containing at least one C2-C6 alcohol, and at least one penetration enhancer.

In another aspect, said process comprises the step of adding 4-OHT in the desired amount, and mixing.

In another aspect, said process comprises the step of adding at least one gelling agent, and mixing.

In another aspect, said process comprises the step of adding at least one aqueous vehicle, and mixing.

In another aspect, said process comprises the steps of:
preparing a mixture containing at least one C2-C6 alcohol, and at least one penetration enhancer;
adding 4-OHT in the desired amount, and mixing;
adding at least one gelling agent, and mixing again; and
adding at least one aqueous vehicle, and mixing again.

The skilled person is familiar with such steps and corresponding techniques, and therefore, would know how to proceed to carry out said process.

The advantages of the invention will become apparent from the following examples, which are given below as mere illustrations, and are non limitative.

The skilled person will appreciate that the present invention can incorporate any number of the preferred features described above.

All citations mentioned herein are hereby incorporated by reference in their entirety.

Other embodiments of the present invention are not presented here, which are obvious to those skilled in the art, and thus are within the scope and the spirit of the present invention. The practice of the present invention will employ, unless otherwise indicated, conventional techniques of pharmacology and pharmaceutics, which are within the skill of the art.

### EXAMPLES

### Example 1: Preparation of Pharmaceutical Compositions (Gels)

Different pharmaceutical compositions were prepared:

| | Quantity per 100 g of gel | | |
|---|---|---|---|
| | 57 mg 4- | 114 mg 4- | 228 mg 4- |
| | OHT Gel: | OHT Gel: | OHT Gel: |
| | 0.057% of | 0.114% of | 0.228% of |
| Ingredient | 4-OHT | 4-OHT | 4-OHT |
| 4-Hydroxy Tamoxifen | 0.057 g | 0.114 g | 0.228 g |
| Absolute ethanol | 66.5 g | 66.5 g | 66.5 g |
| EP-USP | | | |
| Isopropyl myristate, | 1 g | 1 g | 1 g |
| EP-USP | | | |
| Hydroxypropylcellulose, | 1.5 g | 1.5 g | 1.5 g |
| EP-USP | | | |
| Phosphate Buffer | q.s. | q.s. | q.s. |
| (pH 7, diluted 1:4) | 100 g | 100 g | 100 g |

EP: European Pharmacopoeia;
USP: US Pharmacopoeia

The 4-OHT containing compositions are manufactured according to a multi-step mixing process.

In general, the penetration enhancer (here, isopropyl myristate) and the alcohol (here, ethanol) are mixed; 4-OHT in the desired amount is then added to the mixture, which is then combined with hydroxypropylcellulose (Klucel HF). At the end of the process, the preparation is mixed with the aqueous vehicle (here a buffered water solution). The finished product is transferred by turbine to the container/closure system. Since the drug product is light sensitive, steps involving the drug substance are performed under inactinic light.

Preparation of an Aqueous Phosphate Buffer, pH 7:

For a 1000 g lot of aqueous buffer at pH 7, 0.85 g of KH2PO4 and 3.46 g of Na2HPO4 are weighed and diluted with 1000 g of purified water. The solution is mixed in a portable mixer for at least 10 minutes.

The primary mixer tank is checked to assure that proper cleaning procedures have been performed.

The alcohol is added to the mixer tank under a vacuum of 800 mbar without mixing.

The tank is charged with isopropyl myristate. The excipient container is rinsed with alcohol.

The inactinic lights are turned on and the room lights are turned off.

The 4-OHT is loaded into the mixer tank and the container is rinsed with alcohol.

The solution is mixed for 20 minutes at 2,000 rpm for the turbine and at 40 rpm for the scraper.

The mixer tank is charged with hydroxypropylcellulose (Klucel HF) under 800 mbar vacuum, with the turbine at 2,000 rpm. The turbine is stopped at the end of the operation.

The material is mixed for 20 minutes using the alternated mode:
Turbine at 2,000 rpm during 20 seconds and 600 rpm during 20 seconds.
Scraper: 40 rpm/min

The turbine is stopped at the end of the operation.

The phosphate buffer is transferred into the mixer tank under 800 mbar vacuum and with the scraper at 40 rpm.

The product is mixed for 20 minutes using the alternated mode:
Turbine: 2,000 rpm during 20 seconds and 600 rpm during 20 seconds.
Scraper: 40 rpm/min

The turbine is stopped at the end of the operation.

The mixer tank is placed gradually until 100 mbar of vacuum:
1st step: 250 mbar
2nd step: 150 mbar
3rd step: 100 mbar

The vacuum is maintained at 100 mbar for 2 minutes with the scraper at 40 rpm.
The product is mixed for 10 minutes with the scraper at 40 rpm. The scraper is stopped at the end of the operation.
The gel is transferred into a stainless steel tank with a cover via extraction turbine to avoid the introduction of additional air.
The sampling is done while the transfer is being performed.

The gels thus obtained are stable, colorless, and transparent to slightly opalescent

### Example 2: In Vitro Absorption Studies

### Material and Methods

### 4-OHT Gels

Radiolabelled 4-OHT (3H) was used in the preparation of pharmaceutical compositions as described above.

Compositions (gels) with 4-OHT concentrations of 0.057%, 0.114% and 0.228% were prepared.

### In Vitro Dermal Absorption:

### Principle

In vitro transdermal absorption is quantitatively studied on human ventral dermatomed biopsies placed in a static diffusion cell (Franz cell), which allows contacting dermis with a survival liquid (receptor fluid) in which absorption through skin is to be dosed.

### Cell

A dermal biopsy is maintained horizontally between two parts of the cell, thus delimiting two compartments:
one epidermal compartment is comprised of a glass cylinder, having a precisely defined area of 1.77 cm², placed on the upper side of the skin;
the other, dermal, applied to the lower face of the tegument, comprises a reservoir of fixed volume carrying a lateral collection port.

The two elements are assembled via a clamp.

The lower compartment (dermal) is filled with a receptor liquid constituted of a sodium chloride solution at 9 g/L supplemented with bovine serum albumin at 15 g/L. At each time point, the survival liquid is entirely sampled out by the lateral collection port and is replaced by fresh liquid.

The lower part of the cell is thermostated at 37° C. Homogeneity of the temperature and the content in the receptor fluid, is maintained by stirring (magnetic stirrer).

The upper part (epidermal compartment) is open towards the exterior, thus exposing the epidermal surface to the air in the laboratory.

### Preparation of Human Ventral Dermatomed Skin Dermal Biopsies:

These are samples from human ventral skin from plastic surgery from white donors. Skin is kept at −20° C. before use. Adherent sub-dermal fat is removed with a scalpel, and skin is brought to a thickness of about 0.5 mm with a dermatome. For each formulation tested and each volume, 12 franz cells are set up and 3 different donors skin samples are equally distributed between the 12 cells.

### General Protocol

Franz cells are usually installed the day before loading the formulation to be studied. The epidermal compartment is contacted with the atmosphere in the laboratory, the dermal compartment is thermostated to 37° C. and the skin is contacted with albuminated physiological serum for about 17 hours.

The desired amount of gel (5 μL, 10 μL or 20 μL) is applied with a micropipette onto the whole of the surface of the epidermis delimited by the glass cylinder. Sampling from the liquid contained in the dermal compartment is carried out via the lateral collection port at time point 24 h.

### Radioactivity Measurements

Detection is carried out by liquid scintillation using a particle counter Packard-tricarb 2900 TR.

### Preparation of Radioactive Samples:

The receptor liquid sampled from the lower compartment of the diffusion cells is directly incorporated in 15 mL of liquid scintillation cocktail (Picofluor 40R, Packard) and metered for radioactivity measurement.

### Radioactivity Measurements:

Metering rate is corrected, as far as quenching is concerned, by the method of the external calibration, in order to obtain disintegrations per minute (dpm) accounting for the real activity of each sample. The background is deducted for each sample in cpm. For each scintillation liquid, a specific quenching curve is established.

Results are expressed in weight or percentage of substance found in the samples with respect to the administered amount, determined from the metering rates of suitably diluted calibrations.

### Protocol

For a given concentration in 4-OHT in the composition and a given volume of composition applied onto the skin, average results are given, which correspond to 11-12 experimental determinations and are associated to the standard deviation (Sd).

### Results

### In Vitro 4-OHT Penetration in Franz Cells

Average values +/−Sd of 4-OHT amounts in receptor liquid at 24 h:

| Formulation | in % of the applied dose | | Amount in ng | |
|---|---|---|---|---|
| Dosage/volume | average | Sd | average | Sd |
| 57 mg/5 μL | 0.146 | 0.068 | 3.154 | 1.442 |
| 57 mg/10 μL | 0.352 | 0.224 | 15.900 | 10.170 |
| 57 mg/20 μL | 0.788 | 0.371 | 74.312 | 35.012 |
| 114 mg/5 μL | 0.249 | 0.244 | 10.491 | 10.802 |
| 114 mg/10 μL | 0.382 | 0.255 | 34.105 | 23.587 |
| 114 mg/20 μL | 0.995 | 0.762 | 185.592 | 144.420 |
| 228 mg/5 μL | 0.258 | 0.170 | 20.300 | 13.908 |
| 228 mg/10 μL | 0.313 | 0.180 | 56.048 | 33.006 |
| 228 mg/20 μL | 0.908 | 0.603 | 339.058 | 224.372 |

For example, in the above table, “114 mg/10 μL” means application on a skin surface of 1.77 cm² of 10 μL of a gel formulation, wherein said gel formulation contains 114 mg of 4-OHT per 100 g of gel (0.114% of 4-OHT).

The above results show that the amount of absorbed 4-OHT is far better controlled with the 0.114% and 0.228% compositions than with the 0.057% composition for the same amount of 4-OHT applied:

For the 0.057% composition, when increasing 4-fold the application volume (from 54, to 20 μL), the percentage of absorption is unexpectedly increased about 5-fold. This results in a 23.5-fold increase in terms of absorbed amount of 4-OHT, although the applied amount of 4-OHT is only increased 4-fold (the application volume having been increased from 5 to 20 μl). In other terms, when increasing 4-fold the applied amount of 4-OHT, the actual absorbed amount is multiplied by a factor 23.5.

As a conclusion, with the 0.057% composition, there is a great variability in the amount of absorbed 4-OHT, and the absorption is not linear with the volume applied. This effect is surprisingly decreased with the 0.114% and 0.228% compositions.

For example, with the 0.228% composition, increasing 4-fold the application volume (from 5 μL to 20 μL) results in a more modest increase in the 4-OHT absorption percentage (about 3-fold) and the increase in the amount of absorbed 4-OHT is only 16.6-fold.

This feature is particularly advantageous where the compositions of the invention are to be applied onto the breast:

The above described experiment shows that the amount of absorbed 4-OHT varies to a lesser extent as a function of volume with the 0.114% and 0.228% compositions, as compared to the 0.057% composition.

The above experiments are all conducted on the same skin surface area (1.77 cm²). Hence this protocol (varying the volume of gel application for a same surface area) reflects what would happen when applying a same amount of gel onto varying skin surface areas. This is precisely the issue when the compositions are to be applied onto breast skin.

Advantageously, the compositions of the invention allow an improved compliance by the patient: for the same therapeutic dose to be applied (higher doses), with the 0.228% and the 0.114% compositions, the amount of composition/gel to be applied is decreased, thus permitting a daily application, instead of bi-daily. As could be expected, it can be seen that applying the same amount of 4-OHT (either applying 10 μl of the 0.114% formulation or applying 5 μl of the 0.228% formulation), results in a similar amount of absorbed 4-OHT (34±24 versus 20±14; p>0.05, Mann Whitney). However, in view of the non linear increased absorption with a higher volume of the 0.057% formulation as discussed above, applying the same amount of 4-OHT using 20 μL of the 0.057% formulation or using 10 μL of the 0.114% formulation results in significantly different amounts of absorbed 4-OHT (74±35 versus 34±24; p=0.004, Mann Whitney); similarly, applying the same amount of 4-OHT using 20 μL of the 0.057% formulation or using 5 μL of the 0.228% formulation results in significantly different amounts of absorbed 4-OHT (74±35 versus 20±14; p=0.001, Mann Whitney). Thus, it can be seen that patients with smaller breasts, who are to apply the 0.057% formulation under a thick layer (equivalent to 20 μL per 1.77 cm² Franz cell), are overexposed as compared to patients applying the same formulation in a same amount/volume on a larger skin surface area. Patients with smaller breasts would therefore benefit from the formulations according to the present invention, as the compositions of the invention provide better compliance.

### Example 3: In Vivo Absorption Studies

### Protocol

The present studies are open-label studies of various dose levels of 4-OHT in 32 healthy women with a regular menstrual cycle. The women are randomized to one of the doses (daily doses of 1 mg 4-OHT for groups A and B, 2 mg 4-OHT for group C, or 4 mg 4-OHT for group D), which they apply to their breasts each morning for 21 consecutive days.

4-OHT is formulated as a 0.228% w/w hydroalcoholic gel (daily doses of 4-OHT studied: 1 mg (group B) or 4 mg (group D)) or as a 0.057% w/w hydroalcoholic gel (daily doses of 4-OHT studied: 1 mg (group A) or 2 mg (group C)), as described in Example 1.

The time of maximum plasma concentration (tmax), the maximum plasma concentration (Cmax), the area under the plasma concentration versus time curve until 24 hours and until the last measurable plasma concentration (AUCO-24 and AUCtlast) and extrapolated until infinity (AUCO-8) are assessed on the day of last dosing (Day 21).

### Results

After multiple dosing of each of the 1 mg, 2 mg and 4 mg doses of 4-OHT, steady state is reached between Day 14 and Day 20.

The maximum concentration after the last dose is reached on average at 12.8, 6.0, 11.8 and 3.5 hours after dosing respectively for the 1 mg (0.057% gel), 1 mg (0.228% gel), 2 mg (0.057% gel) and 4 mg (0.228% gel) dose groups. However, individual tmax values are highly variable and ranged from 0 to 24 hours. The high variability in tmax reflects the flatness of the concentration time profiles (with no clear peak concentrations).

Cmax (in pg/ml) and AUC 0-24 (in pg*hr/ml) are provided in the table below.

| | A | B | C | D |
|---|---|---|---|---|
| | 1 mg 4-OHT | 1 mg 4-OHT | 2 mg 4-OHT | 4 mg 4-OHT |
| | gel 0.057% = | gel 0.228% = | gel 0.057% = | gel 0.228% = |
| Group | 1.76 g of gel | 0.44 g of gel | 3.5 g of gel | 1.76 g of gel |
| Cmax | 35 ± 22 | 20 ± 9 | 52 ± 32 | 149 ± 99 |
| AUC 0-24 | 619 ± 466 | 383 ± 177 | 998 ± 653 | 2810 ± 1850 |

Gel 0.057% = gel containing 0.057% 4-OHT
Gel 0.228% = gel containing 0.228% 4-OHT

The following comments can be made on these results:
Absorption of 4-OHT (as represented by AUC) is dose-proportional between the 2 mg dose and the 4 mg dose, taking into account the variability (see results for group C versus those for group D); since it is not possible to apply a daily dose of 4 mg with a single application/day of the 0.057% gel due to a very large amount of gel (7 g on both breasts), the 0.228% gel advantageously provides a proper amount of gel and an absorption (2810 pg*hr/ml, group D) which is similar to the theoretical absorption of the less concentrated gel (2*998 pg*hr/ml, twice the absorption for group C); this is in agreement with the in vitro results above, showing that for an adequate amount of gel, (10 and 5 μl), similar 4-OHT absorptions can be achieved between gels (absorption is proportional to the dose);

Although dose proportionality is observed above, there is unexpectedly no linearity between the 1 mg and the 4 mg 4-OHT doses for the 0.228% gel (2810 pg*hr/ml divided by 4≠383 pg*hr/ml; see results for group B versus those for group D: although the dose for group D is 4-fold that of group B, the absorption for group D is not 4-fold that of group B); this is in agreement with the in vitro data showing the influence of gel amount on absorption, demonstrating that excessive amounts of gel influence absorption in a non-linear fashion; however, in the present case, the amount of gel applied on the breasts are not excessive; thus, these in vivo data provide surprising results as far as very small amounts of gel are concerned (0.44 g);
A consequence of the above results is that absorption is not comparable for the same therapeutic dose of 1 mg for the two gels (0.057% and 0.228%) applied on a finite area (the breast); this means that a 4-fold increase in the amount of gel/cm² significantly influence absorption; this was surprising and totally unexpected, as contradictory results have been observed with a very similar gel containing testosterone instead of 4-OHT: the application of a given dose of this testosterone gel on one site versus four sites, resulting into a variation of the amount of gel/cm² of 4 fold, did not significantly affect the testosterone absorption. (Wang C & al, “Pharmacokinetics of Transdermal testosterone gel in hypogonadal men: application of gel at one site versus four sites: a general clinical research center study” The journal of Clinical Endocrinology & Metabolism, 2000, vol 85, n° 3, p 964-969).

1. A pharmaceutical composition comprising:
0.228% 4 hydroxy tamoxifen,
50-75% at least one C2-C6 alcohol,
0.1-5.0% at least one gelling agent,
0.1-5.0% at least one penetration enhancer, and
20-50% an aqueous vehicle,
wherein percentages (%) are weight to weight of the composition.

2.-3. (canceled)

4. The pharmaceutical composition of claim 1, wherein said C2-C6 alcohol is selected from ethanol, propan-1-ol and propan-2-ol, and mixtures of two or more thereof.

5. The pharmaceutical composition of claim 1, wherein said gelling agent is selected from acrylic acid-based polymer gelling agents, cellulosic gelling agents, and mixtures thereof.

6. The pharmaceutical composition of claim 1, wherein said gelling agent comprises at least one gelling agent selected from carboxymethylcelluloses, hydroxypropylcelluloses, hydroxyethylcelluloses, ethylcelluloses, hydroxymethylcelluloses, hydroxypropylmethylcelluloses, and mixtures of two or more thereof.

7. The pharmaceutical composition of claim 1, wherein said penetration enhancer comprises isopropyl myristate.

8. The pharmaceutical composition of claim 1, wherein said aqueous vehicle comprises at least one base.

9. The pharmaceutical composition of claim 8, wherein said base is selected from triethanolamine, sodium hydroxide, ammonium hydroxide, potassium hydroxide, arginine, aminomethylpropanol, tromethamine, and mixtures of two or more thereof.

10. The pharmaceutical composition of claim 1, wherein said aqueous vehicle comprises at least one buffer.

11. The pharmaceutical composition of claim 10, wherein said buffer is selected from citrate, tris maleate, phosphate buffers, and mixtures of two or more thereof.

12. The pharmaceutical composition of claim 10, wherein the composition has a pH of from 7.5 to 10.0.

13. The pharmaceutical composition of claim 1, which is in the form of a gel formulated for delivering 4-hydroxy tamoxifen transdermally or transcutaneously.

14. A dose packet, unit dose packet or multiple dose packet containing the pharmaceutical composition of claim 1.

15. A dispenser containing the pharmaceutical composition of claim 1.

16. The dispenser of claim 15, wherein the dispenser comprises a hand pump.

17. A method for preparing the pharmaceutical composition of claim 1, comprising preparing a mixture that contains at least one C2-C6 alcohol, at least one penetration enhancer, 4-hydroxy tamoxifen, at least one gelling agent and at least one aqueous vehicle.

18. A method of treating or preventing a condition selected from benign breast disease, scarring, keloids, gynecomastia, breast cancer, mastalgia, and conditions involving dense breast tissue, comprising administering to a patient in need thereof the pharmaceutical composition of claim 1.

19. A pharmaceutical composition comprising:
0.228% 4-hydroxy tamoxifen,
50-75% ethanol,
0.1-5.0% hydroxypropylcellulose,
from 0.1-5.0% isopropyl myristate, and
phosphate buffer,
wherein percentages (%) are weight to weight of the composition.

20. The pharmaceutical composition of claim 19, comprising:
0.228% 4-hydroxy tamoxifen,
66.5% ethanol,
1.5% hydroxypropylcellulose,
1% isopropyl myristate, and
phosphate buffer,
wherein percentages (%) are weight to weight of the composition.
